(19) United States
(12) Patent Application Publication
(10) Pub. No.: US 2021/0355276 A1
(43) Pub. Date: Nov. 18, 2021

(54) Ketals and Polyketals as Release Agents

(71) Applicant: The Trustees of Columbia University in the City of New York, New York, NY (US)

(72) Inventors: Chathuranga C. DE SILVA, New York, NY (US); Porakrit LEOPHAIRATANA, New York, NY (US); Jeffrey T. KOBERSTEIN, New York, NY (US)

(21) Appl. No.: 17/194,414

(22) PCT Filed: Sep. 16, 2019
(86) PCT No.: PCT/US2019/051257

§ 371 Date: Mar. 8, 2021

Related U.S. Application Data
(60) Provisional application No. 62/732,384, filed on Sep. 17, 2018.

(51) Int. Cl. C08G 65/332; A61K 47/60; A61K 47/54; A61K 8/86; A61K 45/06; C08G 65/331

(52) U.S. Cl. CPC C08G 65/3322; A61K 47/60; C08G 65/3311; A61K 8/86; A61K 45/06; A61K 47/54

(57) ABSTRACT

Disclosed herein are ketal compounds, oligomers, and polyketals that are obtained in both high purity and high yield. These ketals and polyketals are utilized for their ability to readily release small chemical molecules, preferably fragrance molecules. Also disclosed are the utility of ketals and polyketals as delivery vehicles for controlled release of fragrances over time and/or on demand.

### CROSS REFERENCE TO RELATED APPLICATIONS

This application is an International PCT Application which claims priority to U.S. Provisional Patent Application No. 62/732,384, filed on Sep. 17, 2018, all of which is incorporated herein by reference in its entirety.

### DESCRIPTION

This disclosure relates to ketals and polyketals. In particular, this disclosure relates to ketal and polyketal adducts and/or compounds, polymers, macromolecules and/or oligomers that are utilized for their ability to readily release small chemical molecules, where an adduct is a product of a direct addition of two or more distinct molecules, resulting in a single reaction product containing all atoms of all components and the result is considered a distinct molecular species. More specifically, this disclosure also provides for utilizing these ketals as encapsulants for controlled release of fragrances. In a preferred aspect, these ketals, polyketal and polymer-ketal adduct encapsulants provide functionalization for mono and poly alcohol-based fragrances for controlled/delayed released under acidic stimuli.

### BACKGROUND

Fragrances, which are volatile molecules which emanate a scent, are attractive for applications in toiletries, cosmetics, and home care products. These scent generating molecules impart scents when provided to the human body, animals, objects, and living spaces, impart favorable olfactory effects. While some volatility of fragrances is essential for human sensory response, their highly volatile nature often limits the duration of delivery of the fragrance from a product or when otherwise applied to a substrate, i.e., a topical application.

There is a continuing search for materials having useful perfumery fragrance characteristics which can be bio-compatible. These materials are sought either as replacements for naturally occurring compounds or as completely new scents or odors in their own right. For practical reasons such materials should possess other favorable properties, e.g. substantivity and stability in applications, all in addition to their useful fragrances/odors.

Several methods have been developed to mitigate the volatility of fragrance(s). One approach, so-called profragrance, involves covalent binding of volatile fragrance to substrates to obtain non-volatile compounds, which can be released only upon external stimuli; for instance, some deodorants contain profragrances that are released upon exposure to moisture. However, this approach is normally limited to fragrances with chemical functionalities such as aldehydes or ketones which react with water molecules and is not always possible or effective.

A further alternative method is via encapsulation of fragrance molecules as “core” surrounded by a capsule or “shell”, the latter being a total or partial barrier to the delivery of the fragrance molecules until one or more conditions are satisfied, i.e., physical breakage or a change in pH or solubilization of the shell.

Conventional encapsulation techniques, such as interfacial polymerization, complex coacervation, and sol-gel encapsulation, are utilized to encapsulate fragrance in microcapsules. In forming such microencapsulated fragrances, fragrance molecules are usually first dispersed and emulsified in an, aqueous phase, and thereafter an encapsulating material is added to the emulsion which ideally forms shells about the dispersed droplets of the fragrance molecules in the emulsion. All too often however, such techniques lack highly efficient encapsulation and retention of fragrance within the microcapsules. Since many fragrances are mostly amphiphilic alcohols (hydrophobic while exhibiting partial water solubility), substantial loss can occur during the emulsification of fragrance in the aqueous phase prior to encapsulation. Furthermore, these techniques may result in microcapsules with non-uniform dimensions, i.e, excessive variances in their size, shell thickness, or structures. Such techniques limit optimization of protective shell design which effectively encapsulate and retain the fragrance Indeed, for fragrance molecules with molecular weights less than 300 Da, high mobility of fragrances can lead to rapid leakage through the shell wall and, consequently, to significant loss of encapsulated fragrance during storage and thus prior to any end use with an associated product.

Thus, there is a real need in the art for fragrance materials which provide for improved delivery characteristics for fragrance molecules, and for methods for producing such materials. There is also a need in the art for fragrance materials which provide for the controlled delivery of volatile fragrance molecules from a substrate to which the said fragrance materials are applied, or from a product of which the said fragrance materials form a part.

Therefore, there is also an unmet need for a strategy that remediates amphiphilicity of volatile fragrances as well as enables controlled release of the fragrance upon demand with non-toxic bio-compatible substances. These and other needs in the art are met by the use of the ketal and polyketal chemistries of the present disclosure, particularly as per preferred embodiments to deliver volatile fragrance molecules in a controllable manner

There also exists a need in the art for bio-sourced compounds as semi-encapsulants which can release small chemical molecules including pharmaceuticals and fragrances. It is also desirable that such materials be synthesized economically in large volumes. A still further advantage would be manufacture of high yield and high purity ketals and polyketals that meet these requirements. It is also known that PEG-ketals (linkers) are attractive alternatives due to their cost effectiveness, mild reaction conditions to accomplish synthesis, excellent solubility in most organic solvents of interest, high conjugation capacity and most of the ketal chemistries are GRAS (Generally Recognized As Safe) by the FDA. One of the limitations known is the difficulty in driving the necessary ketal reactions to completion. This disclosure also provides pathways for overcoming these limitations.

### SUMMARY

The present disclosure provides for polymer-alcohol-based-fragrance-conjugates that are linked via acid labile ketal linkers, and which release alcohol-based fragrance from the conjugate in the presence of acid stimuli. These labile ketal linkers can be made from with alcohol functional polymers, oligomers and small molecules including but not limiting to polysaccharides (e.g., starch, modified starch, cellulose, hydroxypropyl methylcellulose, hydroxyethyl cellulose), monosaccharides, lipids, polyester, polyamides, polyvinyl alcohol, polynucleotides, polyacetals, polyurethanes. In addition, it is possible to prepare polyketals to accomplish essentially the same task(s). In all these cases, degradation products formed as a result of the acid stimulus are, 1) the alcohol-based fragrance in its original form, 2) remnants of the polymers used to form the polymer-alcohol-based-fragrance conjugate, and in some cases, 3) acetone. An important feature is that acetaldehyde is not formed during the degradation as in the case of polyacetals. In the fragrance industry, acetaldehyde is considered hazardous and, therefore, its use is avoided. The polyketal and PEG polymers are preferably, low molecular weight molecules of no greater than 50 chain lengths (degree of polymerization (DP)<50) resulting in weight average molecular weights ranging from 1,000-25,000 g/mol. Higher molecular weight molecules and/or with higher degrees of polymerization are nonetheless also possible.

In an aspect of the invention, fragrance materials are formed utilizing organic polymers such as polyolefins, polyalkylene glycols such as polyethylene glycol (PEG), polypropylene glycol, celluloses (both naturally occurring and modified) and other polymers which can be functionalized with one or more ketone groups, preferably keto end groups, and subsequently reacted with at least an alcohol moiety containing fragrance molecules to form a bond between the said fragrance molecule with at least one of the said keto-functionalized polymers. The keto-functionalized polymers may be based on homopolymers which may be optionally substituted with other atoms and can also be based on copolymers comprising monomeric units which include one or more of the above identified organic polymers. The keto-functionalized polymers may be linear or branched, and may also be cyclic or include intermediate cyclic portions intermediate ends of a polymer chain. Additionally it is to be recognized that the invention also encompasses the use of a keto-functionalized organic polymer including those listed above which includes one or more ketone moieties intermediate the ends of the polymer molecule; such one or more ketone moieties are reactive with at least one of the alcohol moieties of the one or more alcohol-moiety containing fragrance molecules between the ends thereof, and in such an instance, the functionalized organic polymer which may or may not include one or more of the ketone end groups; thus keto-functionalized polymers which lack one or more terminal keto groups are within the scope of the present invention as well, as one or more ketone moieties present within the chain of the polymer molecule and intermediate the terminal ends thereof may provide one or more satisfactory reaction sites for the alcohol moiety containing fragrance molecule.

Suitable alcohol moiety containing fragrance molecules include those having at least one reactive alcohol moiety which is reactive with at least one keto-functionalized organic polymer. Suitable alcohol moiety containing fragrance molecules include mono-, di-, tri- and further poly-alcohol moieties, which at least one alcohol moiety is present within the same fragrance molecule. Such alcohol moiety containing fragrance molecules may be interchangeably referred to merely as “alcohol containing fragrance molecules” with or without a reference to the number of reactive alcohol moieties, i.e., “mono-alcohol” refers to a fragrance molecule having a single reactive alcohol moiety within the fragrance molecule.

With respect to the formation of the polymer-alcohol-fragrance-conjugates, it is at least required that a keto-functionalized organic polymer react with at least one alcohol moiety containing fragrance molecule having one or more reactive alcohol moieties to form a bond therebetween, preferably a covalent bond. Examples of such reactions are disclosed in further detail further in this specification. In certain preferred two mono-alcohol containing fragrance molecules are reacted with a polymer having two keto-functional end groups to form a bond at each end between a mono-alcohol containing fragrance molecule and the keto-functionalized polymer. In further and preferred embodiments, a mono-alcohol or poly-alcohol (i.e, diol, triol, etc.) containing fragrance molecule is reacted with a keto-functionalized polymer, at keto functional groups present within the polymer molecule, which may be one or more keto functional groups at one or more ends of the polymer molecule or anywhere intermediate the ends thereof. In further and preferred embodiments, a mono-alcohol or poly-alcohol (i.e, diol, triol, etc.) containing fragrance molecule is reacted with at least two keto-functionalized polymers, at keto functional groups present within each of the polymer molecules, which may be one or more keto functional groups at one or more ends of the polymer molecule or anywhere intermediate the ends thereof; such a reaction provides for the formation of polymer chains or networks wherein the reacted fragrance molecule forms intermediate linkages between adjacent polymer molecules, and specifically include configurations wherein a fragrance molecule provides an intermediate link between ends of two polymer molecules, or between an end of a polymer molecule and a second polymer molecule which may be at an end thereof or at any other keto functional site within the second polymer molecule. Such a reaction may also be used to form polymer chains or polymer networks comprising intermediate fragrance molecules concurrently linked to two or more polymer chains; i.e, wherein a triol-alcohol fragrance molecule is use, up to three polymer molecules may be concurrently reacted and liked to the said fragrance molecule, and logically, poly-alcohol fragrance molecules with more available reactive alcohol moieties may be reactive with a correspondingly higher number of keto-functionalized polymers. It is also understood that certain of the foregoing reactions will form a cyclic structure wherein two keto-functional groups of a keto-functionalized polymer chain react with different reactive alcohol moieties of a poly-alcohol containing fragrance molecule; a preferred embodiment of such a reaction being a keto-functionalized polymer having both end groups functionalized which end groups react with two reactive alcohol moieties of a fragrance molecule, which would force cyclization of the polymer molecule.

In the first of several embodiments, synthetic routes are used for the formation of the polymer-alcohol-fragrance-conjugates and subsequent release of fragrance molecules from the conjugate using the degradable ketal linkers. Each of the routes considered are cost effective and use inert starting materials and have non-toxic degradation products. In one embodiment, the invention provides one or more polyethylene glycol (PEG) linked ketals having a structure (I) (also referred to as a “polymer-alcohol-based-fragrance conjugate”) represented as:

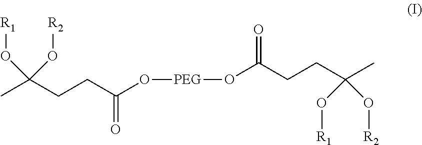

wherein R₁, R₂ are either the same or different derivatives of molecules/macromolecules with alcohol functionality(ies). Each of the R₁, R₂ groups of structure (I) may be different from any other R₁, R₂ groups present in the molecule, or more specifically each of these groups may be unique and different from any other or all of these groups within the molecule.

In a further embodiment the present invention includes one or more polyethylene glycol (PEG) linked ketals (also referred to as a “polymer-alcohol-based-fragrance conjugate”) having a structure (Ia) represented as:

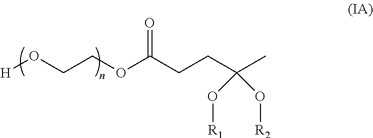

wherein R₁, R₂ are either the same or different derivatives of molecules/macromolecules with alcohol functionality(ies). Each of the R₁, R₂ groups of structure (IA) may be different from any other R₁, R₂ groups present in the molecule, or more specifically each of these groups may be unique and different from any other or all of these groups within the molecule.

In the foregoing molecules of structure (I) and/or structure (IA), R₁, R₂ each independently may be selected from one or more of alcohol derivatives from the group consisting of;
hydroxy cinnamyl alcohol;
rhodinol;
anisyl alcohol;
alpha-terpinol;
nerol;
maltol;
leaf alcohol;
ebanol;
dihydromercinol;
hydroxycitronellal;
lavender ketone;
raspberry ketone;
dimetol;
phenyl ethyl alcohol;
alpha-methylcinnamic alcohol;
linalool oxide;
acetoin;
isopentyl alcohol;
isoamyl alcohol;
2-phenyl methanol;
4-allyl-2-methoxyphenol (eugenol);
3-(2-bornyloxy)-2-methyl-1-propanol;
2-tert-butylcyclohexanol;
4-tert-butylcyclohexanol;
benzyl alcohol;
1-decanol;
9-decen-1-ol;
dihydroterpineol;
2,4-dimethyl-4-cyclohexen-1-yl methanol;
2,4-dimethylcyclohexyl methanol;
2,6-dimethyl-2-heptanol;
2,6-dimethyl-4-heptanol;
3a,4,5,6,7,7a-hexahydro-2,4-dimethyl-4,7-methano[1H] inden-5-ol;
3,7-dimethyl-1,6-nonadien-3-ol;
2,6-dimethyl-2,7-octadien-6-ol (linalool);
cis-3,7-dimethyl-2,6-octadien-1-ol (nerol);
trans-3,7-dimethyl-2,6-octadien-1-ol (geraniol;
3,7-dimethyl-1,7-octanediol;
3,7-dimethyl-1-octanol (tetrahydrogeraniol);
2,6-dimethyl-2-octanol (tetrahydromyrcenol);
3,7-dimethyl-3-octanol (tetrahydrolinalool);
2,6-dimethyl-7-octen-2-ol (dihydromyrcenol);
3,7-dimethyl-6-octen-1-ol (citronellol);
2,2-dimethyl-3-(3-methylphenyl)-1-propanol;
2,2-dimethyl-3-phenyl-1-propanol, 2-ethoxy-4-methoxymethylphenol;
2-ethyl-4-(2,2,3-trimethyl-3-cyclopenten-1-yl)-2-buten-1-ol;
cis-3-hexen-1-ol, 4-(4-hydroxy-3-methoxyphenyl)-2-butanone;
1-hydroxy-2-(1-methyl-1-hydroxyethyl)-5-methylcyclohexane;
3-(hydroxymethyl)-2-nonanone;
4-(4-hydroxy-4-methylpentyl)-3-cyclohexene-1-carboxaldehyde;
isoborneol;
3-isocamphylcyclohexanol;
2-isopropenyl-5-methylcyclohexanol (isopulegol);
1-isopropyl-4-methylcyclohex-3-enol (terpinenol);
4-isopropylcyclohexanol, 1-(4-isopropylcyclohexyl) ethanol;
4-isopropylcyclohexylmethanol;
2-isopropyl-5-methylcyclohexanol (menthol);
2-isopropyl-5-methylphenol (thymol), 5-isopropyl-2-methylphenol (carvacrol);
2-(4-methyl-3-cyclohexenyl)-2-propanol (terpineol);
2-(4-methylcyclohexyl)-2-propanol (dihydroterpineol);
4-methoxybenzyl alcohol, 2-methoxy-4-methylphenol;
3-methoxy-5-methylphenol;
1-methoxy-4-propenylbenzene (anethol);
2-methoxy-4-propenylphenol (isoeugenol);
4-methyl-3-decen-5-ol;
2-methyl-6-methylene-7-octen-2-ol (myrcenol);
3-methyl-4-phenyl-2-butanol;
2-(2-methylphenyl) ethanol;
2-methyl-4-phenyl-1-pentanol;
3-methyl-5-phenyl-1-pentanol;
2-methyl-1-phenyl-2-propanol;
(1-methyl-(1,2,2-trimethylbicyclo[3.1.0]hex-3-ylmethyl) cyclopropyl) methanol;
3-methyl-4-(2,2,6-trimethylcyclohexen-1-yl)-2-butanol;
2-methyl-4-(2,2,3-trimethyl-3-cyclopenten-1-yl)-2-buten-1-ol;
(3-methyl-1-(2,2,3-trimethyl-3-cyclopentenyl)-3-cyclohexen-1-yl) methanol;
3-methyl-5-(2,2,3-trimethyl-3-cyclopenten-1-yl)-4-penten-2-ol;
2-methyl-2-vinyl-5-(1-hydroxy-1-methylethyl) tetrahydrofuran;
trans,cis-2,6-nonadienol;
1-nonanol;
nopol;
1,2,3,4,4a,5,6,7-octahydro-2,5,5-trimethyl-2-naphthol;
1-octanol;
3,4,5,6,6-pentamethyl-2-heptanol;
2-phenylethanol;
2-phenylpropanol;
3-phenylpropanol (hydrocinnamic alcohol);
3-phenyl-2-propen-1-ol (cinnamic alcohol);
4-(5,5,6-trimethylbicyclo[2.2.1]hept-2-yl) cyclohexan-1-ol;
3,5,5-trimethylcyclohexanol;
2,4,6-trimethyl-4-cyclohexen-1-ylmethanol;
5-(2,2,3-trimethyl-3-cyclopentenyl)-3-methylpentan-2-ol;
3,7,11-trimethyl-2,6,10-dodecatrien-1-ol (farnesol);
3,7,11-trimethyl-1,6,10-dodecatrien-3-ol (nerolidol);
3,5,5-trimethyl-1-hexanol (isononanol);
1-undecanol;
10-undecen-1-ol; and
vetiverol.

These R₁, R₂ moieties may mono-alcohol or poly-alcohol (i.e., diol-, triol-) derivatives.

In certain embodiments the R₁, R₂ moieties are of the same type, i.e, the same moiety. In other embodiments the R₁, R₂ moieties are of two types, i.e., are of only two different moieties. In still other embodiments, the R₁, R₂ moieties illustrated on structure (I) and (Ia) are three or four different moieties.

In addition, the R₁ and R₂ in either of structure (I) and/or (Ia) be selected from the group consisting provitamins, vitamins, pain relief agents, and small molecule pharmaceuticals, as distinguished from mono-alcohol or poly-alcohol containing fragrance moieties.

In an embodiment of the present invention, R₁, R₂ are both substituted 2-phenylethanol units which results in a 2-phenylethanol substituted ketal structure represented in the following structure (II);

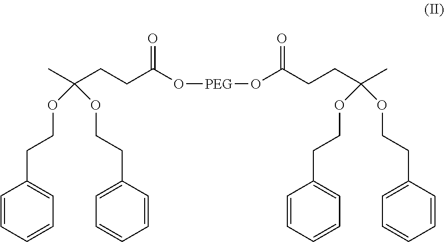

In addition, the R₁, R₂ moieties can both be substituted isoamyl alcohol units that result in a isoamyl alcohol substituted ketal structure represented in the following structure (III);

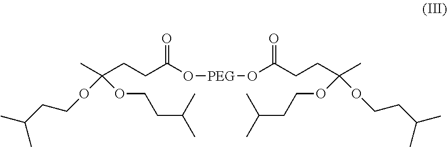

Here, the 2-phenylethanol substituted ketal reaches at least 90 percent conversion in a reaction between methyl levulinate and 2-phenylethanol that occurs in a presence of tetrabutylammonium tribromide (TBAB) and trimethyl orthoformate (TMOF).

In other embodiments, the isoamyl alcohol substituted ketal reaches at least 50 percent conversion, more preferably at least 80, and most preferably at least 90 percent conversion in a reaction between methyl levulinate and isoamyl alcohol that occurs in a presence of tetrabutylammonium tribromide (TBAB) and trimethyl orthoformate (TMOF). An aspect of the present invention is a process for producing a polyethylene glycol (PEG) linked ketal in two steps:

(i) reacting a polyethylene glycol with a levulinic acid together with 1-ethyl, 3,(3-dimethylamino propyl) carbodiimide (“EDC”), 4-dimethyl amino pyridine (“DMAP”), and dichloromethane (“DCM”) to form PEG linked ketals and, preferably subsequently,
(ii) reacting alcohol functional fragrance molecules (preferably, mono-alcohol functional fragrance molecules) with the PEG linked ketal in a presence of tetrabutylammonium tribromide (TBAB) and trimethyl orthoformate (TMOF), which function as catalysts, to produce fragranced PEG linked ketals (also referred to as a “polymer-alcohol-based-fragrance conjugates”) according to Structure IA; the foregoing process (reaction) scheme is illustrated as follows;

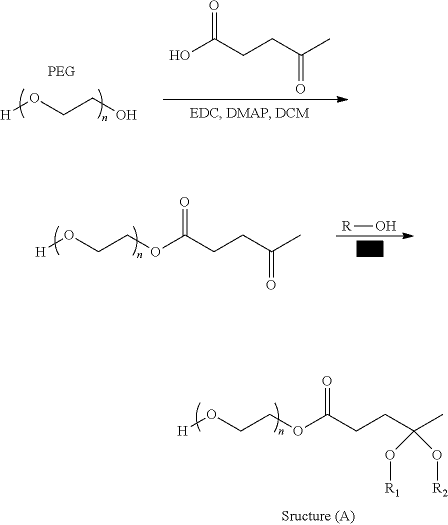

In the foregoing reaction R₁, R₂ are either identical or different mono-alcohols fragrance derivatives, and/or identical or different poly-alcohol fragrance derivatives or as described above

Also, the R₁ and R₂ moieties can be selected from the group consisting of identical or different provitamins, vitamins, pain relief agents, and small molecule pharmaceuticals as distinguished from mono-alcohol and poly-alcohol containing fragrance moieties.

A further aspect of the invention the acid hydrolysis (“decomplexation”) of fragranced PEG linked ketals (“polymer-alcohol-based-fragrance conjugate”) according to the following reaction scheme:

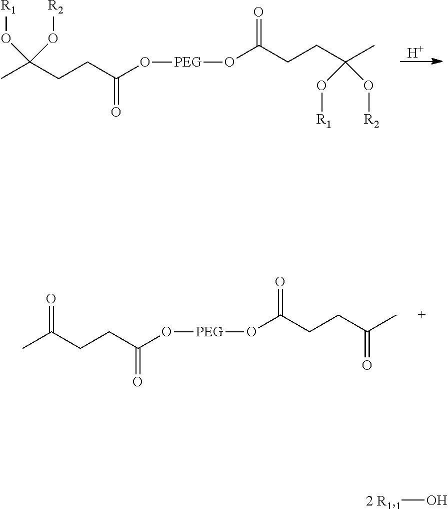

wherein PEG linkaged ketal structures (as may have been formed according to the foregoing process described herein; see Structure I) are acid catalyzed at a pH of about 7 or less, preferably less than 7 and as a result of this catalysis releases an alcohol-based fragrance and ketone as by-products as a result of said acid hydrolysis. The acid hydrolysis requires only that the fragranced PEG linked ketals (“polymer-alcohol-based-fragrance conjugate”) be contacted in an environment wherein the pH is about 7 or less, preferably at a pH of less than 7 which induces the de-complexation of the polymer-alcohol-based-fragrance conjugate (i.e., Structure I) and release of the alcohol based R₁, R₂ molecules, preferably which are fragrance molecules. In the decomplexation process, when the PEG linkaged ketal is contacted with an acid, the PEG linkaged ketal structure undergoes an acid catalysis which releases the one or more fragrance molecules having at least one alcohol moiety and, in some cases, also acetone as a by-product.

A further aspect of the invention is the acid hydrolysis (“decomplexation”) of fragranced PEG linked ketals (“polymer-alcohol-based-fragrance conjugate”) according to the following reaction scheme:

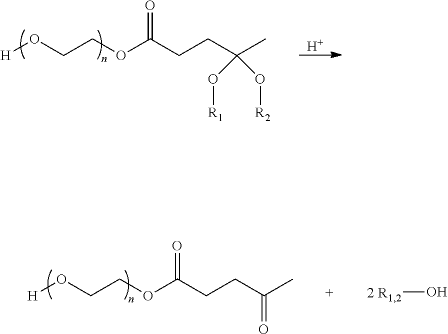

wherein PEG linkaged ketal structures (as may have been formed according to the foregoing process described herein; see Structure IA) are acid catalyzed at a pH of about 7 or less, preferably less than 7 and as a result of this catalysis releases an alcohol-based fragrance and ketone as by-products as a result of said acid hydrolysis. The acid hydrolysis requires only that the fragranced PEG linked ketals (“polymer-alcohol-based-fragrance conjugate”) be contacted in an environment wherein the pH is about 7 or less, preferably at a pH of less than 7 which induces the de-complexation of the polymer-alcohol-based-fragrance conjugate (i.e., Structure IA) and release of the alcohol based R₁, R₂ molecules, preferably which are fragrance molecules. In the decomplexation process, when the PEG linkaged ketal is contacted with an acid, the PEG linkaged ketal structure undergoes an acid catalysis which releases the one or more fragrance molecules having at least one alcohol moiety and, in some cases, also acetone as a by-product.

In yet another embodiment, the present invention one or more polyketals (“polymer-alcohol-based-fragrance conjugate”) comprising a structure (Structure B) represented as:

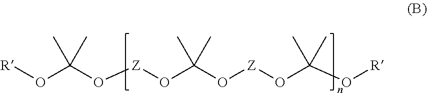

wherein
R′ are each a terminated independent hydrogen, C₁-C₁₀ alkyl alcohol, or C₅-C₆ cycloalkyl alcohol that is optionally substituted with an oxygen in the ring and/or further optionally substituted with one or more aryl groups;
Z is a C₁-C₁₀ alkyl, and/or a C₅-C₆ cycloalkyl including cyclohexane, that is optionally substituted with an oxygen in the ring and/or further optionally substituted with one or more aryl groups such that O—Z—O is an ester group in that it is derived from an acid in which at least one —OH (hydroxyl) group is replaced by an —O-alkyl (alkoxy) or —O-aryl group;
and where n is in a range between 1-200.

Here, acid catalysis of the polyketal (Structure (B)) with an acid at a pH of about 7, preferably below 7 releases acid catalyzed degradation substituents according to the following reaction scheme;

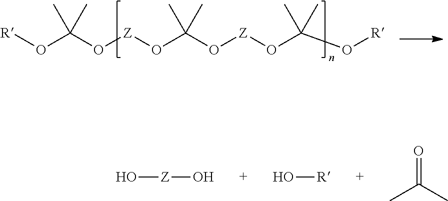

R′ is a terminated independent hydrogen, or R′ is a C₁-C₁₀ alkyl, or C₅-C₁₀ cycloalkyl and wherein R′—OH is a C₁-C₁₀ alkyl alcohol or a C₅-C₁₀ cycloalkyl alcohol and R′ of R′—OH is not H and wherein;
Z of the substituents is a C₁-C₁₀ alkyl, and/or a C₅-C₆ cycloalkyl including cyclohexane, that is optionally substituted with an oxygen in the ring and/or further optionally substituted with one or more aryl groups such that O—Z—O is an ester group in that it is derived from an acid in which at least one —OH (hydroxyl) group is replaced by an —O-alkyl (alkoxy) or —O-aryl group.

The polyketals (Structure (B)) include R′ groups which may be the same or different and which are selected from one or more of;
hydroxy cinnamyl alcohol
rhodinol
anisyl alcohol
alpha-terpinol
nerol
maltol
leaf alcohol
ebanol
dihydromercinol
hydroxycitronellal
lavender ketone
raspberry ketone
dimetol
phenyl ethyl alcohol
alpha-methylcinnamic alcohol
linalool oxide
acetoin
isopentyl alcohol
isoamyl alcohol
2-phenyl methanol
4-allyl-2-methoxyphenol (eugenol);
3-(2-bornyloxy)-2-methyl-1-propanol;
2-tert-butylcyclohexanol;
4-tert-butylcyclohexanol;
benzyl alcohol;
1-decanol;
9-decen-1-ol;
dihydroterpineol;
2,4-dimethyl-4-cyclohexen-1-yl methanol;
2,4-dimethylcyclohexyl methanol;
2,6-dimethyl-2-heptanol;
2,6-dimethyl-4-heptanol;
3a,4,5,6,7,7a-hexahydro-2,4-dimethyl-4,7-methano[1H] inden-5-ol;
3,7-dimethyl-1,6-nonadien-3-ol;
2,6-dimethyl-2,7-octadien-6-ol (linalool);
cis-3,7-dimethyl-2,6-octadien-1-ol (nerol);
trans-3,7-dimethyl-2,6-octadien-1-ol (geraniol;
3,7-dimethyl-1,7-octanediol;
3,7-dimethyl-1-octanol (tetrahydrogeraniol);
2,6-dimethyl-2-octanol (tetrahydromyrcenol);
3,7-dimethyl-3-octanol (tetrahydrolinalool);
2,6-dimethyl-7-octen-2-ol (dihydromyrcenol);
3,7-dimethyl-6-octen-1-ol (citronellol);
2,2-dimethyl-3-(3-methylphenyl)-1-propanol;
2,2-dimethyl-3-phenyl-1-propanol, 2-ethoxy-4-methoxymethylphenol;
2-ethyl-4-(2,2,3-trimethyl-3-cyclopenten-1-yl)-2-buten-1-ol;
cis-3-hexen-1-ol, 4-(4-hydroxy-3-methoxyphenyl)-2-butanone;
1-hydroxy-2-(1-methyl-1-hydroxyethyl)-5-methylcyclohexane;
3-(hydroxymethyl)-2-nonanone;
4-(4-hydroxy-4-methylpentyl)-3-cyclohexene-1-carboxaldehyde;
isoborneol;
3-isocamphylcyclohexanol;
2-isopropenyl-5-methylcyclohexanol (isopulegol);
1-isopropyl-4-methylcyclohex-3-enol (terpinenol);
4-isopropylcyclohexanol, 1-(4-isopropylcyclohexyl) ethanol;
4-isopropylcyclohexylmethanol;
2-isopropyl-5-methylcyclohexanol (menthol);
2-isopropyl-5-methylphenol (thymol), 5-isopropyl-2-methylphenol (carvacrol);
2-(4-methyl-3-cyclohexenyl)-2-propanol (terpineol);
2-(4-methylcyclohexyl)-2-propanol (dihydroterpineol);
4-methoxybenzyl alcohol, 2-methoxy-4-methylphenol;
3-methoxy-5-methylphenol;
1-methoxy-4-propenylbenzene (anethol);
2-methoxy-4-propenylphenol (isoeugenol);
4-methyl-3-decen-5-ol;
2-methyl-6-methylene-7-octen-2-ol (myrcenol);
3-methyl-4-phenyl-2-butanol;
2-(2-methylphenyl) ethanol;
2-methyl-4-phenyl-1-pentanol;
3-methyl-5-phenyl-1-pentanol;
2-methyl-1-phenyl-2-propanol;
(1-methyl-(1,2,2-trimethylbicyclo[3.1.0]hex-3-ylmethyl) cyclopropyl) methanol;
3-methyl-4-(2,2,6-trimethylcyclohexen-1-yl)-2-butanol;
2-methyl-4-(2,2,3-trimethyl-3-cyclopenten-1-yl)-2-buten-1-ol;
(3-methyl-1-(2,2,3-trimethyl-3-cyclopentenyl)-3-cyclohexen-1-yl) methanol;
3-methyl-5-(2,2,3-trimethyl-3-cyclopenten-1-yl)-4-penten-2-ol;
2-methyl-2-vinyl-5-(1-hydroxy-1-methylethyl) tetrahydrofuran;
trans,cis-2,6-nonadienol;
1-nonanol;
nopol;
1,2,3,4,4a,5,6,7-octahydro-2,5,5-trimethyl-2-naphthol;
1-octanol;
3,4,5,6,6-pentamethyl-2-heptanol;
2-phenylethanol;
2-phenylpropanol;
3-phenylpropanol (hydrocinnamic alcohol);
3-phenyl-2-propen-1-ol (cinnamic alcohol);
4-(5,5,6-trimethylbicyclo[2.2.1]hept-2-yl) cyclohexan-1-ol;
3,5,5-trimethylcyclohexanol;
2,4,6-trimethyl-4-cyclohexen-1-ylmethanol;
5-(2,2,3-trimethyl-3-cyclopentenyl)-3-methylpentan-2-ol;
3,7,11-trimethyl-2,6,10-dodecatrien-1-ol (farnesol);
3,7,11-trimethyl-1,6,10-dodecatrien-3-ol (nerolidol);
3,5,5-trimethyl-1-hexanol (isononanol);
1-undecanol;
10-undecen-1-ol; and
vetiverol.

The present invention also includes a process creating fragrance releasing polyketals (Structure (B)) according to the following reaction scheme:

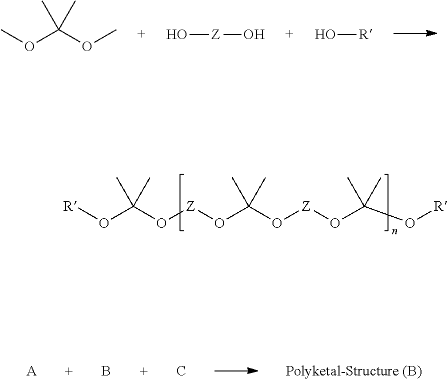

wherein in this case, a diol (B) is reacted with 2,2-dimethoxypropane and a mono-alcohol fragrance (HO—R′) in the presence of p-toluene-sulfonic acid (C) to provide the polyketal (“polymer-alcohol-based-fragrance conjugate”) of Structure (B);

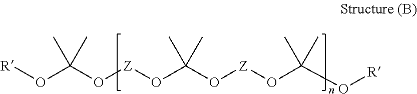

wherein R′ are each a terminated independent hydrogen, C₁-C₁₀ alkyl, or C₅-C₆ cycloalkyl that is optionally substituted with an oxygen in the ring and/or further optionally substituted with one or more phenyl groups, or one or both of the R′ groups which may be the same or different and are selected from one or more of;
hydroxy cinnamyl alcohol
rhodinol
anisyl alcohol
alpha-terpinol
nerol
maltol
leaf alcohol
ebanol
dihydromercinol
hydroxycitronellal
lavender ketone
raspberry ketone
dimetol
phenyl ethyl alcohol
alpha-methylcinnamic alcohol
linalool oxide
acetoin
isopentyl alcohol
isoamyl alcohol
2-phenyl methanol
4-allyl-2-methoxyphenol (eugenol);
3-(2-bornyloxy)-2-methyl-1-propanol;
2-tert-butylcyclohexanol;
4-tert-butylcyclohexanol;
benzyl alcohol;
1-decanol;
9-decen-1-ol;
dihydroterpineol;
2,4-dimethyl-4-cyclohexen-1-yl methanol;
2,4-dimethylcyclohexyl methanol;
2,6-dimethyl-2-heptanol;
2,6-dimethyl-4-heptanol;
3a,4,5,6,7,7a-hexahydro-2,4-dimethyl-4,7-methano[1H] inden-5-ol;
3,7-dimethyl-1,6-nonadien-3-ol;
2,6-dimethyl-2,7-octadien-6-ol (linalool);
cis-3,7-dimethyl-2,6-octadien-1-ol (nerol);
trans-3,7-dimethyl-2,6-octadien-1-ol (geraniol;
3,7-dimethyl-1,7-octanediol;
3,7-dimethyl-1-octanol (tetrahydrogeraniol);
2,6-dimethyl-2-octanol (tetrahydromyrcenol);
3,7-dimethyl-3-octanol (tetrahydrolinalool);
2,6-dimethyl-7-octen-2-ol (dihydromyrcenol);
3,7-dimethyl-6-octen-1-ol (citronellol);
2,2-dimethyl-3-(3-methylphenyl)-1-propanol;
2,2-dimethyl-3-phenyl-1-propanol, 2-ethoxy-4-methoxymethylphenol;
2-ethyl-4-(2,2,3-trimethyl-3-cyclopenten-1-yl)-2-buten-1-ol;
cis-3-hexen-1-ol, 4-(4-hydroxy-3-methoxyphenyl)-2-butanone;
1-hydroxy-2-(1-methyl-1-hydroxyethyl)-5-methylcyclohexane;
3-(hydroxymethyl)-2-nonanone;
4-(4-hydroxy-4-methylpentyl)-3-cyclohexene-1-carboxaldehyde;
isoborneol;
3-isocamphylcyclohexanol;
2-isopropenyl-5-methylcyclohexanol (isopulegol);
1-isopropyl-4-methylcyclohex-3-enol (terpinenol);
4-isopropylcyclohexanol, 1-(4-isopropylcyclohexyl) ethanol;
4-isopropylcyclohexylmethanol;
2-isopropyl-5-methylcyclohexanol (menthol);
2-isopropyl-5-methylphenol (thymol), 5-isopropyl-2-methylphenol (carvacrol);
2-(4-methyl-3-cyclohexenyl)-2-propanol (terpineol);
2-(4-methylcyclohexyl)-2-propanol (dihydroterpineol);
4-methoxybenzyl alcohol, 2-methoxy-4-methylphenol;
3-methoxy-5-methylphenol;
1-methoxy-4-propenylbenzene (anethol);
2-methoxy-4-propenylphenol (isoeugenol);
4-methyl-3-decen-5-ol;
2-methyl-6-methylene-7-octen-2-ol (myrcenol);
3-methyl-4-phenyl-2-butanol;
2-(2-methylphenyl) ethanol;
2-methyl-4-phenyl-1-pentanol;
3-methyl-5-phenyl-1-pentanol;
2-methyl-1-phenyl-2-propanol;
(1-methyl-(1,2,2-trimethylbicyclo[3.1.0]hex-3-ylmethyl) cyclopropyl) methanol;
3-methyl-4-(2,2,6-trimethylcyclohexen-1-yl)-2-butanol;
2-methyl-4-(2,2,3-trimethyl-3-cyclopenten-1-yl)-2-buten-1-ol;
(3-methyl-1-(2,2,3-trimethyl-3-cyclopentenyl)-3-cyclohexen-1-yl) methanol;
3-methyl-5-(2,2,3-trimethyl-3-cyclopenten-1-yl)-4-penten-2-ol;
2-methyl-2-vinyl-5-(1-hydroxy-1-methylethyl) tetrahydrofuran;
trans,cis-2,6-nonadienol;
1-nonanol;
nopol;
1,2,3,4,4a,5,6,7-octahydro-2,5,5-trimethyl-2-naphthol;
1-octanol;
3,4,5,6,6-pentamethyl-2-heptanol;
2-phenylethanol;
2-phenylpropanol;
3-phenylpropanol (hydrocinnamic alcohol);
3-phenyl-2-propen-1-ol (cinnamic alcohol);
4-(5,5,6-trimethylbicyclo[2.2.1]hept-2-yl) cyclohexan-1-ol;
3,5,5-trimethylcyclohexanol;
2,4,6-trimethyl-4-cyclohexen-1-ylmethanol;
5-(2,2,3-trimethyl-3-cyclopentenyl)-3-methylpentan-2-ol;
3,7,11-trimethyl-2,6,10-dodecatrien-1-ol (farnesol);
3,7,11-trimethyl-1,6,10-dodecatrien-3-ol (nerolidol);
3,5,5-trimethyl-1-hexanol (isononanol);
1-undecanol;
10-undecen-1-ol; and
vetiverol;

Z is a C₁-C₁₀ alkyl, and/or a C₅-C₆ cycloalkyl including cyclohexane, that is optionally substituted with an oxygen in the ring and/or further optionally substituted with one or more aryl groups such that O—Z—O is an ester group in that it is derived from an acid in which at least one —OH (hydroxyl) group is replaced by an —O-alkyl (alkoxy) or —O-aryl group and where n is in a range between 1-200.

However, the polyketals reach a higher molecular weight by reflux at 100 degrees Celsius to boil off methanol, addition of 2,2-dimethoxypropane and benzene every 2 hours for 12 hours and use of a 5 Angstrom molecular sieve to capture excess methanol. Each of the polyketals can thereby reach a weight average molecular weight (Mw) of greater than 1000 g/mol and exhibit a polydispersity index (PDI) of less than 3.00.

Any of the polymer-alcohol-based-fragrance conjugates (i.e., Structure I, Structure IA, Structure B) may, in preferred embodiments, be de-complexed by contact with parts of a mammalian body, such as the epidermis, or with saliva or other bodily fluid which has a pH of about 7 or less, preferably less than 7. Thus, the polymer-alcohol-based-fragrance conjugates find particular use in personal care products of the type which are to be topically applied such as solid, liquid, semi-solid liquid gel product (i.e., skin cream, deodorant, perfume composition), or which may be ingested into the body, (i.e, orally ingested such as a toothpaste, mouthwash, chewing gum, lozenge). The polymer-alcohol-based-fragrance conjugates may, in preferred embodiments, be de-complexed by coming into contact with a substrate or surface or other inanimate environment having a pH of about 7 or less, preferably a pH of less than 7. Such for example may be hard surface care products such as cleaning, disinfecting or sanitizing products. Such may be consumer products, including without limitation: hard surface treatment compositions, soft surface treatment compositions, any of which may be in a solid, liquid, semi-solid (i.e., gel, viscous liquid) or aersolizable product format.

The decomplexation of the polymer-alcohol-based-fragrance conjugates may be controlled, and may occur over a longer duration after their application. For example, when one or more of the polymer-alcohol-based-fragrance conjugates comprising fragrance molecule derivatives are topically applied to parts of the human body, the decomplexation typically occurs over a longer period of time than in the absence of the polymer-alcohol-based-fragrance conjugate. This gives a time-delay release benefit to the fragrance molecule derivatives, as decomplexation of the fragrance molecule derivatives may occur over the course of several hours and thus provides a more durable benefit. Another important advantage of the use of a fragrance molecule derivatives is wherein the complexed fragrance molecule derivatives are based on “high” notes, which a typically relatively highly volatile fragrance molecules which are notoriously fleeting with regard to their organoleptic effect. When such relatively highly volatile fragrance molecules are delivered via a polymer-alcohol-based-fragrance conjugates as described herein, subsequent to being contacted in an acidic environment, e.g., in a dermal topical product application, their release is slowed or retarded for a much longer interval of time than could be realized using a like composition without the polymer-alcohol-based-fragrance conjugates. Such a benefit is not only advantageous for personal care products, but also find advantage in other products including hard and soft surface treatment compositions as well as air care and air treatment products and compositions.

### BRIEF DESCRIPTION OF THE DRAWINGS

FIG. 1 is an NMR confirmation of the presence of methyl levulinate.

FIG. 2 is a Structure IB of Isoamyl alcohol (IAA) substituted PEG linked ketal.

FIG. 3 is a GPC chromatogram of polyketal (Mw=2900 g·mol⁻¹; 1.65) end-functionalized with 2-Phenylethanol.

FIG. 4 is a ¹H-NMR (400 MHz) spectra for polyketal end-functionalized with 2-Phenylethanol in CDCl₃.

The present disclosure is further illustrated and described by the following Detailed Description and Examples.

### DETAILED DESCRIPTION

Keto-functionlizable polymers useful in the present invention, include poly(ethylene glycol) (PEG), cellulose, and also include most polymer systems (preferably with hydroxyl (—OH) end groups) can be functionalized by using molecules containing ketone groups that are subsequently reacted with mono- or poly-alcohol containing fragrance molecules so to incorporate these fragrance molecules into these polymers, which form complexes, which may also be referred do as polymer-alcohol-based-fragrance conjugates, which may be subsequently decomplexed when exposed to appropriate conditions. Such polymers include oligomeric and polymeric chains exhibiting ketal linkages. The preferred polyethylene glycol (PEG) system described herein, demonstrates ketalization chemistry using ketones, and in this demonstration, a “two-armed” a ketone-polyethylene glycol-levulinic acid (PEG-LA) is formed via a one-step synthesis via carbodiimide by coupling of the PEG with levulinic acid resulting in a 98.9% recovery yield. A preferred PEG molecular weight range is about 1,000-25,000 g/mol. While a ketone-polyethylene glycol-levulinic acid (PEG-LA) is a preferred embodiment of the invention, it is a non-limiting example and it is understood that other functionalized PEG's may be used as well according to the present invention, i.e, including those known to the art and disclosed in US Patent Application 2017/0362380, the complete contents of which are hereby incorporated by reference.

To form the polymer-alcohol-based-fragrance conjugates the keto-functionalized ketone-polyethylene PEG-LA (levulinic acid) was further reacted with mono-alcohols, here, fragrances bearing 2-phenylethanol and isoamyl alcohol to produce PEG-ketals As previously noted, as well as described in more detail herein, the use of other fragrance molecules having one, two, three of more reactive alcohol moieties are clearly contemplated as forming part of the invention. In order to use the PEG-ketal product(s) as release agents (or as “delivery vehicle”), the PEG-ketals are reacted under a suitable environmental condition, i.e., exposed to an acidic pH, and the alcohol containing fragrances are released by degradation (or decomplexation of the polymer-alcohol-based-fragrance conjugate), which was validated by qualitative techniques including the detection by perception of the alcohol containing fragrance molecule by the human nose, which is effective in perceiving fragrances at parts per trillion levels or even lower.

A preferred synthesis, mechanism, and resulting structures scheme to form these PEG-ketal compounds (“polymer-alcohol-based-fragrance conjugate”) are provided in the two step fragrance incorporation process shown below;

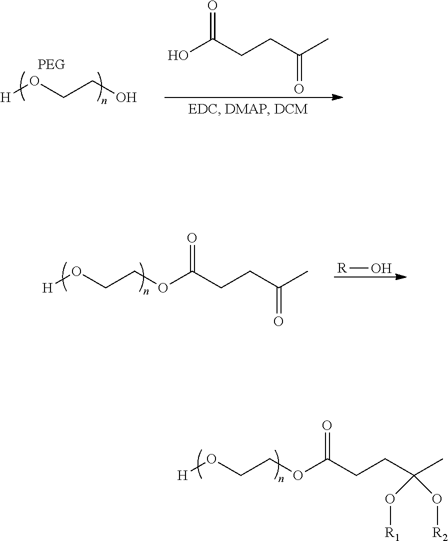

wherein:
DMAP is 4-dimethyl amino pyridine,
EDC is 1-ethyl, 3,(3-dimethylamino propyl) carbodiimide,
TNATB is tetrabutylammonium tribromide and TMOF is trimethyl orthoformate,
DCM is dichloromethane,
and R₁, R₂ are either identical or different derivatives of molecules/macromolecules with alcohol functionality(ies), which may be mono-alcohols, diols, triols or further higher order polyols. Each of the R₁, R₂ groups may be different from any other R₁, R₂ groups present in the resultant molecule, or more specifically each of these groups may be unique and different from any other or all of these groups within the resultant molecule; in certain preferred embodiments each of the R₁, R₂ present are one or more of alcohol derivatives from the group consisting of;
hydroxy cinnamyl alcohol;
rhodinol;
anisyl alcohol;
alpha-terpinol;
nerol;
maltol;
leaf alcohol;
ebanol;
dihydromercinol;
hydroxycitronellal;
lavender ketone;
raspberry ketone;
dimetol;
phenyl ethyl alcohol;
alpha-methylcinnamic alcohol;
linalool oxide;
acetoin;
isopentyl alcohol;
isoamyl alcohol;
2-phenyl methanol;
4-allyl-2-methoxyphenol (eugenol);
3-(2-bornyloxy)-2-methyl-1-propanol;
2-tert-butylcyclohexanol;
4-tert-butylcyclohexanol;
benzyl alcohol;
1-decanol;
9-decen-1-ol;
dihydroterpineol;
2,4-dimethyl-4-cyclohexen-1-yl methanol;
2,4-dimethylcyclohexyl methanol;
2,6-dimethyl-2-heptanol;
2,6-dimethyl-4-heptanol;
3a,4,5,6,7,7a-hexahydro-2,4-dimethyl-4,7-methano[1H] inden-5-ol;
3,7-dimethyl-1,6-nonadien-3-ol;
2,6-dimethyl-2,7-octadien-6-ol (linalool);
cis-3,7-dimethyl-2,6-octadien-1-ol (nerol);
trans-3,7-dimethyl-2,6-octadien-1-ol (geraniol;
3,7-dimethyl-1,7-octanediol;
3,7-dimethyl-1-octanol (tetrahydrogeraniol);
2,6-dimethyl-2-octanol (tetrahydromyrcenol);
3,7-dimethyl-3-octanol (tetrahydrolinalool);
2,6-dimethyl-7-octen-2-ol (dihydromyrcenol);
3,7-dimethyl-6-octen-1-ol (citronellol);
2,2-dimethyl-3-(3-methylphenyl)-1-propanol;
2,2-dimethyl-3-phenyl-1-propanol, 2-ethoxy-4-methoxymethylphenol;
2-ethyl-4-(2,2,3-trimethyl-3-cyclopenten-1-yl)-2-buten-1-ol;
cis-3-hexen-1-ol, 4-(4-hydroxy-3-methoxyphenyl)-2-butanone;
1-hydroxy-2-(1-methyl-1-hydroxyethyl)-5-methylcyclohexane;
3-(hydroxymethyl)-2-nonanone;
4-(4-hydroxy-4-methylpentyl)-3-cyclohexene-1-carboxaldehyde;
isoborneol;
3-isocamphylcyclohexanol;
2-isopropenyl-5-methylcyclohexanol (isopulegol);
1-isopropyl-4-methylcyclohex-3-enol (terpinenol);
4-isopropylcyclohexanol, 1-(4-isopropylcyclohexyl) ethanol;
4-isopropylcyclohexylmethanol;
2-isopropyl-5-methylcyclohexanol (menthol);
2-isopropyl-5-methylphenol (thymol), 5-isopropyl-2-methylphenol (carvacrol);
2-(4-methyl-3-cyclohexenyl)-2-propanol (terpineol);
2-(4-methylcyclohexyl)-2-propanol (dihydroterpineol);
4-methoxybenzyl alcohol, 2-methoxy-4-methylphenol;
3-methoxy-5-methylphenol;
1-methoxy-4-propenylbenzene (anethol);
2-methoxy-4-propenylphenol (isoeugenol);
4-methyl-3-decen-5-ol;
2-methyl-6-methylene-7-octen-2-ol (myrcenol);
3-methyl-4-phenyl-2-butanol;
2-(2-methylphenyl) ethanol;
2-methyl-4-phenyl-1-pentanol;
3-methyl-5-phenyl-1-pentanol;
2-methyl-1-phenyl-2-propanol;
(1-methyl-(1,2,2-trimethylbicyclo[3.1.0]hex-3-ylmethyl) cyclopropyl) methanol;
3-methyl-4-(2,2,6-trimethylcyclohexen-1-yl)-2-butanol;
2-methyl-4-(2,2,3-trimethyl-3-cyclopenten-1-yl)-2-buten-1-ol;
(3-methyl-1-(2,2,3-trimethyl-3-cyclopentenyl)-3-cyclohexen-1-yl) methanol;
3-methyl-5-(2,2,3-trimethyl-3-cyclopenten-1-yl)-4-penten-2-ol;
2-methyl-2-vinyl-5-(1-hydroxy-1-methylethyl) tetrahydrofuran;
trans,cis-2,6-nonadienol;
1-nonanol;
nopol;
1,2,3,4,4a,5,6,7-octahydro-2,5,5-trimethyl-2-naphthol;
1-octanol;
3,4,5,6,6-pentamethyl-2-heptanol;
2-phenylethanol;
2-phenylpropanol;
3-phenylpropanol (hydrocinnamic alcohol);
3-phenyl-2-propen-1-ol (cinnamic alcohol);
4-(5,5,6-trimethylbicyclo[2.2.1]hept-2-yl) cyclohexan-1-ol;
3,5,5-trimethylcyclohexanol;
2,4,6-trimethyl-4-cyclohexen-1-ylmethanol;
5-(2,2,3-trimethyl-3-cyclopentenyl)-3-methylpentan-2-ol;
3,7,11-trimethyl-2,6,10-dodecatrien-1-ol (farnesol);
3,7,11-trimethyl-1,6,10-dodecatrien-3-ol (nerolidol);
3,5,5-trimethyl-1-hexanol (isononanol);
1-undecanol;
10-undecen-1-ol; and
vetiverol.

A further aspect of the invention is the process for decomplexation of the PEG-ketal compounds (viz., “polymer-alcohol-based-fragrance conjugates”) and consequent release of one or more fragrance molecules having at least one alcohol moiety, via an acid hydrolysis process step, as shown below:

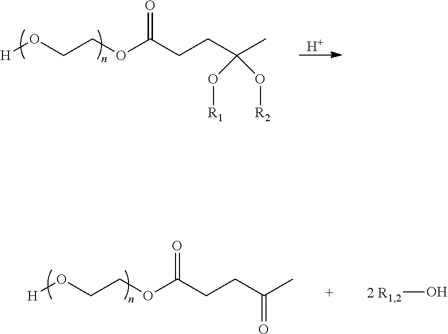

When the PEG linkaged ketal structure is subjected to an environment where the conditions are acidic, or when contacted with an acid, the PEG linkaged ketal structure undergoes an acid catalysis which releases the one or more fragrance molecules having at least one alcohol moiety and, in some cases, also acetone as a by-product. Here, while the foregoing illustration shows a reaction wherein two monohydric alcohol comprising fragrance molecules are released to the ambient environment, it is nonetheless to be understood that these alcohol containing fragrance molecules may also be polyhydric, and further that R₁, R₂ may be identical or different. The foregoing illustrative reaction also demonstrates a delivery system wherein a volatile fragrance molecule having at least one hydroxyl moiety is decomplexed and released to the ambient environment.

In separate embodiments, R₁ and R₂ are identical or different diols and/or polyols, preferably diols or polyols of fragrance molecules. The following Ketal Structure IA is the result of identical R₁ and R₂ substituents reacted with 2-phenylethanol.

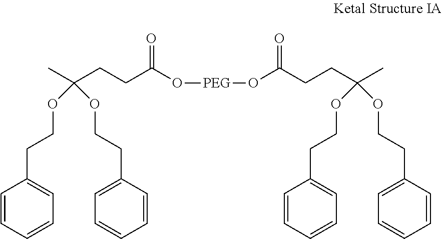

An alternative embodiment includes providing an alternate ketal structure, with IAA (isoamyl alcohol) as the mono-alcohol resulting in Structure IB;

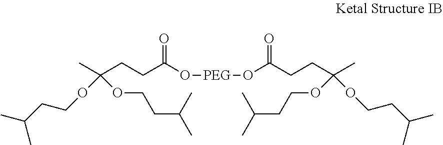

The resultant polyethylene glycol (PEG) linked ketals having a structure (I) (also referred to as a “polymer-alcohol-based-fragrance conjugate”) prior to acid hydrolysis is as given below;

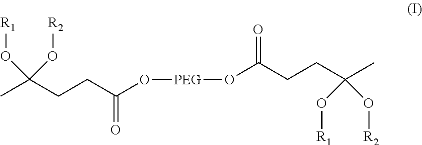

which is a ketal PEG linker structure, wherein R₁, R₂ are either the same or different monoalcohol containing fragrance molecules, and wherein these ketal structures, when acid catalyzed at a pH less than 7, releases the fragrance and acetone as by-products. As previously stated herein, each of the R₁, R₂ groups of structure (I) may be different from any other R₁, R₂ groups present in the molecule, or more specifically each of these groups may be unique and different from any other or all of these groups within the molecule. Also, as previously stated herein, in the foregoing molecule of structure (I), R₁, R₂ each independently may be selected from one or more of alcohol derivatives, particularly one or more alcohol moiety containing fragrance compounds.

To achieve the polyketal structure II (also referred to as a “polymer-alcohol-based-fragrance conjugate”) shown below, which is a fragrance comprising polyketal, the following reaction scheme was followed;

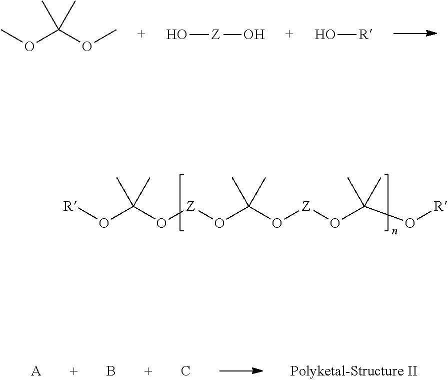

wherein in this case, 1,4 cyclohexanedimethanol (B) is reacted with 2,2-dimethoxypropane and a mono-alcohol fragrance (HO—R′) (C) to provide the polyketal of Structure II;

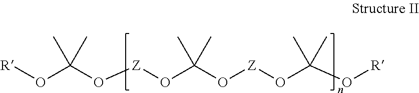

wherein R′ is a terminated independent hydrogen, or R′ is a C₁-C₁₀ alkyl alcohol, or C₅-C₁₀ cycloalkyl and wherein R′—OH is a C₁-C₁₀ alkyl alcohol or a C₅-C₁₀ cycloalkyl alcohol and R′ of R′—OH is not H that is optionally substituted with an oxygen in the ring and/or further optionally substituted with one or more phenyl groups;
Z is a C₁-C₁₀ alkyl, and/or a C₅-C₆ cycloalkyl including cyclohexane, that is optionally substituted with an oxygen in the ring and/or further optionally substituted with one or more aryl groups such that O—Z—O is an ester group in that it is derived from an acid in which at least one —OH (hydroxyl) group is replaced by an —O-alkyl (alkoxy) or —O-aryl group and where n is in a range between 1-200.

When catalyzed with an acid at a pH below 7, the following acid catalyzed degradation substituents are released;

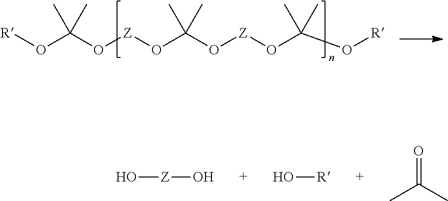

again, wherein R′ is a terminated independent hydrogen, or R′ is a C₁-C₁₀ alkyl, or C₅-C₁₀ cycloalkyl and wherein R′—OH is a C₁-C₁₀ alkyl alcohol or a C₅-C₁₀ cycloalkyl alcohol and R′ of R′—OH is not H that is optionally substituted with an oxygen in the ring and/or further optionally substituted with one or more aryl groups;
Z is a C₁-C₁₀ alkyl, and/or a C₅-C₆ cycloalkyl including cyclohexane, that is optionally substituted with an oxygen in the ring and/or further optionally substituted with one or more aryl groups such that O—Z—O is an ester group in that it is derived from an acid in which at least one —OH (hydroxyl) group is replaced by an —O-alkyl (alkoxy) or —O-aryl group;
and where n is in a range between 1-100.

In this case the released substituents include a fragranced mono-alcohol and the other substituents are non-toxic GRAS by-products.

Although the reaction disclosed above refers to fragrance molecules comprising one or more reactive alcohol moieties, it is within the scope of this invention that these molecules comprising one or more reactive alcohol moieties may be molecules such as vitamin, provitamins, and pain relief medications and pharmaceuticals of small and moderate size, which are distinguishable from fragrance molecules comprising one or more reactive alcohol moieties.

The reaction yield for step (i) above resulted in in high (99+%) purity for the difunctional PEG using levulinic acid and the yield was determined to be 98.9%. Fragrance release, where R₁ and R₂ is 2-phenylethanol (2-PE) provided a lower yield of 93.1% of which at least 50% is the fragrance 2-phenylethanol (2-PE). Fragrance release, where R₁ and R₂ is isoamyl alcohol (IAA) resulted in a yield of 94.7% of which at least 50% is the fragrance functional isoamyl alcohol (IAA).

The structure for incorporation of a 2-phenylethanol fragranced alcohol (rose scent) into a polymer-alcohol-based-fragrance conjugate is illustrated by the ketal-linked PEG structure IA provided below;

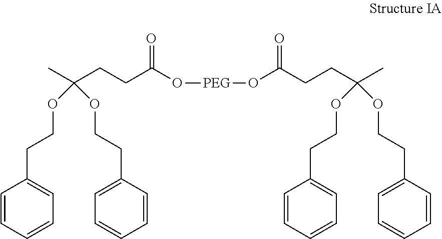

The structure for incorporation of a isoamyl alcohol (IAA) fragranced alcohol (banana scent) into a polymer-alcohol-based-fragrance conjugate is illustrated by in the ketal-linked PEG structure IB provided below;

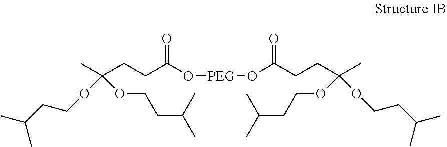

A qualifying small molecule step for the synthesis of both PEG Structures IA and IB was determined to proceed via a route that resulted in essentially 100% yields by utilizing methyl levulinate and levulinic acid. The highest yields along with the fastest kinetics and approx. 100% completion of the reactions utilized a reaction scheme in the presence of tetrabutylammonium tribromide (“TBATB”) and trimethyl orthoformate (“TMOF”) for both of the ketal structures IA and IB from small molecule as illustrated below;

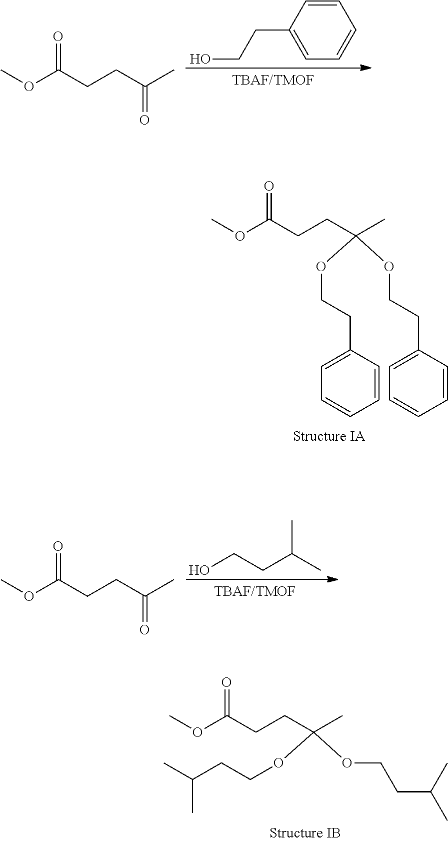

In another embodiment, it was shown that PEG-ketal small molecule conversion efficiency to form the ketal structures IA and IB during the reaction can be improved by lowering the number average molecular, weight, Mw, of the PEG from 6000 to less than 1000. In order to achieve the higher molecular weight polyketals of Structure II, it was necessary to slowly add 2,2-dimethoxypropane and benzene every 2 hours for 12 hours over the course of the reaction, reflux at 100 degrees Celsius to boil away the methanol and to utilize a 5 A molecular sieve to capture the excess methanol.

Certain embodiments of the invention are disclosed in more specific detail in one or more of the following examples. It is to be understood however that these examples are provided solely for the purposes of illustration and not limitation of any aspect of the invention, whose scope is only limited by the claims.

### EXAMPLES

### Example 1. NMR Characterization of the PEG-Ketal

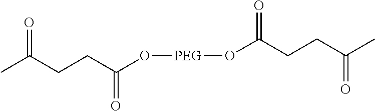

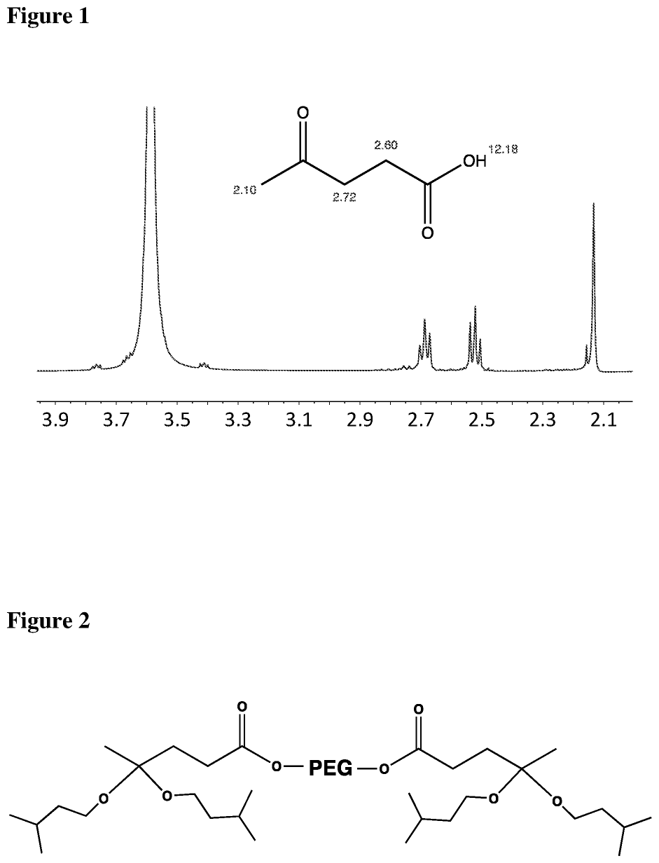

¹H NMR was used to study the conversion efficiency of the esterification of PEG-OH with levulinic acid. FIG. 1 presents NMR confirmation of the presence of methyl levulinate Characteristic peaks at positions δ 2.1, 2.6 and 2.72 shows the presence of levulinic acid. Furthermore, the integration shows the presence for 3H, 2H and 2H which correspond to the theoretical number of protons for each respective peak. The ketone functionality in levulinic acid was used to determine the purity of the product below.

¹H NMR was used to characterize the attachment of isoamyl alcohol (IAA) via ketal linkage as provided in FIG. 2, Structure IB of IAA substituted PEG linked ketal. IAA is an aromatic mono-alcohol that has a fruity fragrance, and is the chemical that constitutes the fragrance in banana. NMR spectrum shows partial disappearance of δ 2.1 and the appearance of peak at δ 1.27. Peak δ 2.1 represents the terminal 3H peak adjacent to the ketone. Upon commencement of reaction, the partial disappearance of peak δ 2.1 indicates ketone is converted to ketal. Appearance of peak 81.27 further confirms this result.

### Example 2. Synthesis of Polyketal

1,4-cyclohexanedimethanol, 2,2-dimethoxypropane, benzene, triethylamine, N,N-dimethylformamide, and deuterated chloroform, tetrahydrofuran, ethyl acetate, and methanol were purchased from commercial suppliers and used as supplied. Additionally, p-Toluene-sulfonic acid was purchased from a commercial supplied but was dried using 5 A molecular sieve in ethyl acetate prior to its use.

The polymer's molecular weight distributions were measured using a Shimadzu LC-10AT Size Exclusion Chromatography (SEC) system equipped with an Agilent guard column and three SEC columns; Shimadzu ultraviolet light (254 nm) and refractive index (RID) detectors; and a Wyatt static light scattering detector. The columns were maintained at 40° C. in a Shimadzu column oven and calibrated using Agilent polystyrene standards in the range of Mp=162-483, 400 g mol⁻¹. THF was used as the mobile phase with the flow rate maintained at 1 mL/min. Proton nuclear magnetic resonance (¹H-NMR) spectra were recorded on a Bruker 400 MHz spectrometer in CDCl₃ with chemical shifts expressed relative to the tetramethylsilane (TMS) standard at δ=0.00.

The scheme below was followed in synthesis of the polyketals;

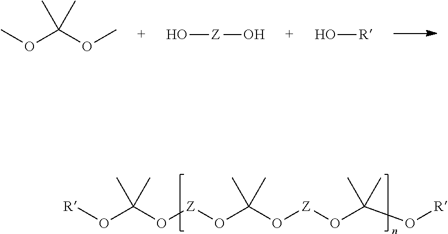

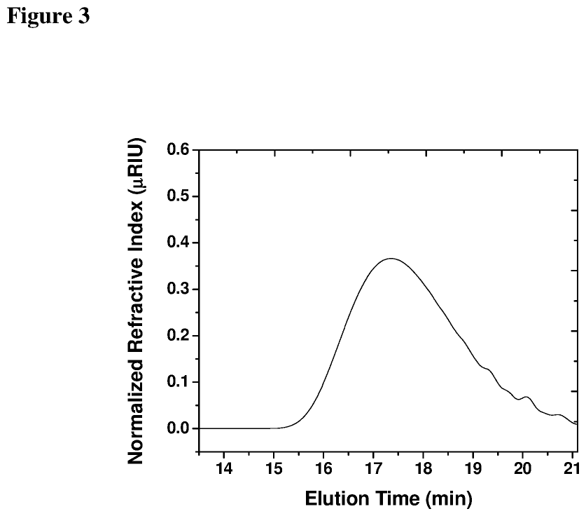

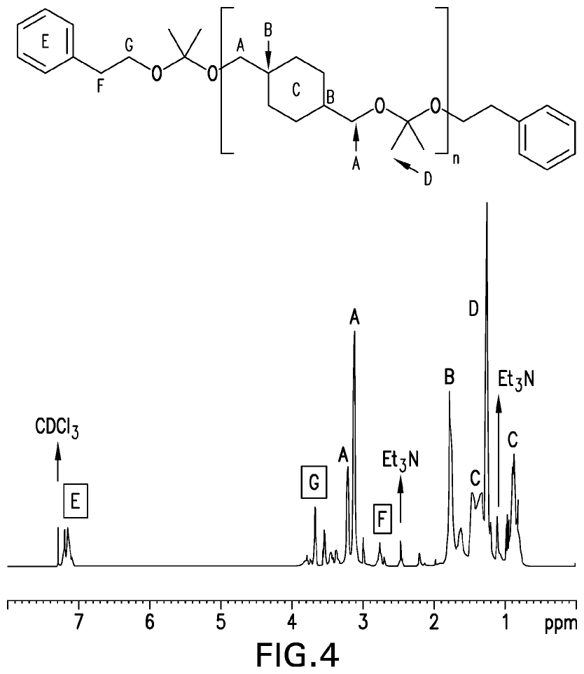

Polyketal copolymers were synthesized in a 100 mL two-necked round bottom flask, connected to a distilling head. The diol, 1,4-cyclohexanedimethanol (12.98 g, 90 mmol) was dissolved in 30 mL of anhydrous benzene and kept at 100° C. Dried 6.82 mL p-toluene-sulfonic acid solution dissolved in ethyl acetate (1.35 mg/mL) was added to the benzene solution. 10 mg activated 5 A molecular sieve was additionally added. The ethyl acetate was distilled off, and the polymerization reaction was initiated by the addition of 2,2-dimethoxypropane (10.94 mL). The reaction was stirred at 100° C. Additional doses of 2,2-dimethoxy-propane (5 mL) and benzene (25 mL) were added slowly to the reaction, every hour for six hours, to compensate for 2,2-dimethoxypropane and benzene that had distilled off. After 1.5 days, 0.5 mL of the fragrance molecule, 2-2-phenylethanol, was added and the reaction solution was stirred additionally at 100° C. for 12 hours. The reaction was stopped with triethylamine (2 mL). The copolymers were purified by under vacuum at 60° C. for 3 days and analyzed by ¹H NMR and GPC as shown in FIG. 3 and FIG. 4.

### Example 3. Polyketal Degradation

The scheme below was utilized in the degradation (“decomplexation”) of the polyketals leading to fragrance release;

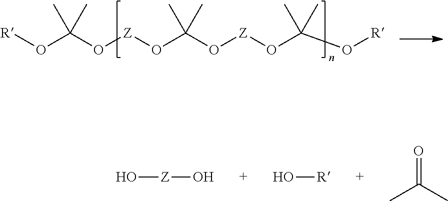

The side chain functionalized polyketal polymers were dissolved in 5 mL THF and added dropwise to 15 mL cold Phosphate Buffer Solution (0.1 M) of pH 7.4 and pH 5.5 (final polymer conc. is 5 g·L⁻¹). The solutions temperature was raised to 37° C. (equivalent to t=0). At various later times, 0.5 ml of each sample was collected, and the degradation was stopped by the addition of excess Et₃N. The solution was immediately injected into a GPC to monitor the molecular weight distribution.

As used herein, the singular forms “a,” “an,” and “the” are intended to include the plural forms as well, unless the context clearly indicates otherwise. “Or” means “and/or.” The endpoints of all ranges directed to the same component or property are inclusive of the endpoint and independently combinable, except when the modifier “between” is used. The modifier “about” used in connection with a quantity is inclusive of the stated value and has the meaning dictated by the context (e.g., includes the degree of error associated with measurement of the particular quantity). A “combination” is inclusive of blends, mixtures, alloys, reaction products, and the like.

In general, the compositions or methods can alternatively comprise, consist of, or consist essentially of, any appropriate components or steps disclosed. The invention can additionally, or alternatively, be formulated so as to be devoid, or substantially free, of any components, materials, ingredients, adjuvants, or species, or steps used in the prior art compositions or that are otherwise not necessary to the achievement of the function and/or objectives of the present claims.

Unless otherwise defined, all terms (including technical and scientific terms) used have the same meaning as commonly understood by one of ordinary skill in the art to which this invention belongs. Compounds are described using standard nomenclature. A dash (“-”) that is not between two letters or symbols is used to indicate a point of attachment for a substituent. For example, CHO is attached through carbon of the carbonyl group. “Alkyl” means a straight or branched chain saturated aliphatic hydrocarbon having the specified number of carbon atoms. “Alkylene” means a straight or branched divalent aliphatic hydrocarbon group having the specified number of carbon atoms. “Aryl” means a cyclic moiety in which all ring members are carbon and a ring is aromatic. More than one ring can be present, and any additional rings can be independently aromatic, saturated or partially unsaturated, and can be fused, pendant, spirocyclic or a combination thereof. “Hetero” means a group or compound including at least one heteroatom (e.g., 1-4 heteroatoms) each independently N, O, S, Si, or P.

A “hydrocarbon group” means a group having the specified number of carbon atoms and the appropriate valence in view of the number of substitutions shown in the structure. Hydrocarbon groups contain at least carbon and hydrogen, and can optionally contain 1 or more (e.g., 1-8, or 1-6, or 1-3) heteroatoms selected from N, O, S, Si, P, or a combination comprising at least one of the foregoing. Hydrocarbon groups can be unsubstituted or substituted with one or more substituent groups up to the valence allowed by the hydrocarbyl group independently selected from a C₁₋₃₀ alkyl, C₂₋₃₀ alkenyl, C₂₋₃₀ alkynyl, C₆₋₃₀ aryl, C₇₋₃₀ arylalkyl, C₁₋₁₂alkoxy, C₁₋₃₀ heteroalkyl, C₃₋₃₀ heteroarylalkyl, C₃₋₃₀ cycloalkyl, C₃₋₁₅ cycloalkenyl, C₆₋₃₀ cycloalkynyl, C₂₋₃₀ heterocycloalkyl, halide (F, Cl, Br, or I), hydroxy, nitro, cyano, amino, azido, amidino, hydrazino, hydrazono, carbonyl, carbamyl, thiol, carboxy (C₁₋₆ alkyl) ester, carboxylic acid, carboxylic acid salt, sulfonic acid or a salt thereof, and phosphoric acid or a salt thereof. While stereochemistry of the various compounds is not explicitly shown, it is to be understood that this disclosure encompasses all isomers.

All cited patents, patent applications, and other references are incorporated by reference in their entirety.

While the invention has been described with reference to exemplary embodiments, it will be understood by those skilled in the art that various changes can be made and equivalents can be substituted for elements thereof without departing from the scope of the invention. In addition, many modifications can be made to adapt a particular situation or material to the teachings of the invention without departing from the essential scope thereof. Therefore, it is intended that the invention not be limited to the particular embodiment disclosed as the mode described and contemplated for carrying out this invention, but that the invention will include all embodiments falling within the scope of the appended claims.

We claim:

1. A polyethylene glycol (PEG) linked ketal comprising a structure represented as: 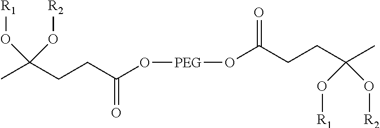
wherein R₁, R₂ are either the same or different derivatives of molecules/macromolecules with alcohol functionality(ies)

2. The PEG linked ketal of claim 1, wherein R₁, R₂ are selected from one or more of alcohol derivatives from the group of alcohols consisting of;
hydroxy cinnamyl alcohol;
rhodinol;
anisyl alcohol;
alpha-terpinol;
nerol;
maltol;
leaf alcohol;
ebanol;
dihydromercinol;
hydroxycitronellal;
lavender ketone;
raspberry ketone;
dimetol;
phenyl ethyl alcohol;
alpha-methylcinnamic alcohol;
linalool oxide;
acetoin;
isopentyl alcohol;
isoamyl alcohol;
2-phenyl methanol;
4-allyl-2-methoxyphenol (eugenol);
3-(2-bornyloxy)-2-methyl-1-propanol;
2-tert-butylcyclohexanol;
4-tert-butylcyclohexanol;
benzyl alcohol;
1-decanol;
9-decen-1-ol;
dihydroterpineol;
2,4-dimethyl-4-cyclohexen-1-yl methanol;
2,4-dimethylcyclohexyl methanol;
2,6-dimethyl-2-heptanol;
2,6-dimethyl-4-heptanol;
3a,4,5,6,7,7a-hexahydro-2,4-dimethyl-4,7-methano[1H] inden-5-ol;
3,7-dimethyl-1,6-nonadien-3-ol;
2,6-dimethyl-2,7-octadien-6-ol (linalool);
cis-3,7-dimethyl-2,6-octadien-1-ol (nerol);
trans-3,7-dimethyl-2,6-octadien-1-ol (geraniol;
3,7-dimethyl-1,7-octanediol;
3,7-dimethyl-1-octanol (tetrahydrogeraniol);
2,6-dimethyl-2-octanol (tetrahydromyrcenol);
3,7-dimethyl-3-octanol (tetrahydrolinalool);
2,6-dimethyl-7-octen-2-ol (dihydromyrcenol);
3,7-dimethyl-6-octen-1-ol (citronellol);
2,2-dimethyl-3-(3-methylphenyl)-1-propanol;
2,2-dimethyl-3-phenyl-1-propanol, 2-ethoxy-4-methoxymethylphenol;
2-ethyl-4-(2,2,3-trimethyl-3-cyclopenten-1-yl)-2-buten-1-ol;
cis-3-hexen-1-ol, 4-(4-hydroxy-3-methoxyphenyl)-2-butanone;
1-hydroxy-2-(1-methyl-1-hydroxyethyl)-5-methylcyclohexane;
3-(hydroxymethyl)-2-nonanone;
4-(4-hydroxy-4-methylpentyl)-3-cyclohexene-1-carboxaldehyde;
isoborneol;
3-isocamphylcyclohexanol;
2-isopropenyl-5-methylcyclohexanol (isopulegol);
1-isopropyl-4-methylcyclohex-3-enol (terpinenol);
4-isopropylcyclohexanol, 1-(4-isopropylcyclohexyl) ethanol;
4-isopropylcyclohexylmethanol;
2-isopropyl-5-methylcyclohexanol (menthol);
2-isopropyl-5-methylphenol (thymol), 5-isopropyl-2-methylphenol (carvacrol);
2-(4-methyl-3-cyclohexenyl)-2-propanol (terpineol);
2-(4-methylcyclohexyl)-2-propanol (dihydroterpineol);
4-methoxybenzyl alcohol, 2-methoxy-4-methylphenol;
3-methoxy-5-methylphenol;
1-methoxy-4-propenylbenzene (anethol);
2-methoxy-4-propenylphenol (isoeugenol);
4-methyl-3-decen-5-ol;
2-methyl-6-methylene-7-octen-2-ol (myrcenol);
3-methyl-4-phenyl-2-butanol;
2-(2-methylphenyl) ethanol;
2-methyl-4-phenyl-1-pentanol;
3-methyl-5-phenyl-1-pentanol;
2-methyl-1-phenyl-2-propanol;
(1-methyl-(1,2,2-trimethylbicyclo[3.1.0]hex-3-ylmethyl) cyclopropyl) methanol;
3-methyl-4-(2,2,6-trimethylcyclohexen-1-yl)-2-butanol;
2-methyl-4-(2,2,3-trimethyl-3-cyclopenten-1-yl)-2-buten-1-ol;
(3-methyl-1-(2,2,3-trimethyl-3-cyclopentenyl)-3-cyclohexen-1-yl) methanol;
3-methyl-5-(2,2,3-trimethyl-3-cyclopenten-1-yl)-4-penten-2-ol;
2-methyl-2-vinyl-5-(1-hydroxy-1-methylethyl) tetrahydrofuran;
trans,cis-2,6-nonadienol;
1-nonanol;
nopol;
1,2,3,4,4a,5,6,7-octahydro-2,5,5-trimethyl-2-naphthol;
1-octanol;
3,4,5,6,6-pentamethyl-2-heptanol;
2-phenylethanol;
2-phenylpropanol;
3-phenylpropanol (hydrocinnamic alcohol);
3-phenyl-2-propen-1-ol (cinnamic alcohol);
4-(5,5,6-trimethylbicyclo[2.2.1]hept-2-yl) cyclohexan-1-ol;
3,5,5-trimethylcyclohexanol;
2,4,6-trimethyl-4-cyclohexen-1-ylmethanol;
5-(2,2,3-trimethyl-3-cyclopentenyl)-3-methylpentan-2-ol;
3,7,11-trimethyl-2,6,10-dodecatrien-1-ol (farnesol);
3,7,11-trimethyl-1,6,10-dodecatrien-3-ol (nerolidol);
3,5,5-trimethyl-1-hexanol (isononanol);
1-undecanol;
10-undecen-1-ol; and
vetiverol.

3. The PEG linked ketal of claim 1 or 2, wherein at least one of R₁, R₂ is a diol derivative.

4. The PEG linked ketal of claim 1 or 2, wherein at least one of R₁, R₂ is a polyol derivative.

5. The PEG linked ketal of any of claims 1-4, wherein each of the R₁, R₂ groups may be unique and different from any other or all of these groups within the PEG linked ketal molecule.

6. The PEG linked ketal of claim 1, wherein R₁ and R₂ are selected from the group consisting of one or more of: provitamins, vitamins, pain relief agents, and small molecule pharmaceuticals, which are as distinguished from mono-alcohol or poly-alcohol containing fragrance moieties.

7. The PEG linked ketal of claim 1, wherein R₁, R₂ are both substituted 2-phenylethanol derivatives within a substituted ketal structure represented as;

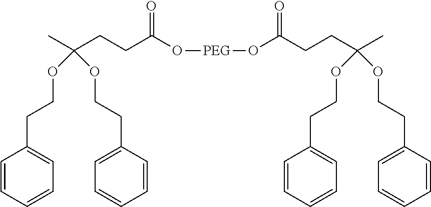

8. The PEG linked ketal of claim 1, wherein R₁, R₂ are both substituted isoamyl alcohol derivatives within a substituted ketal structure represented as;

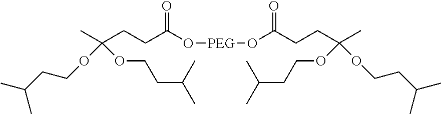

9. A synthesis process for producing a polyethylene glycol (PEG) linked ketal of Structure I 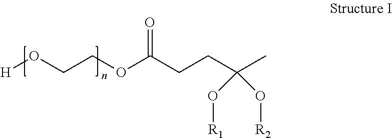
comprising the steps of:
(i) reacting a polyethylene glycol with a levulinic acid together with 1-ethyl, 3,(3-dimethylamino propyl) carbodiimide (EDC), 4-dimethyl amino pyridine (DMAP), and dichloromethane (DCM) to form a PEG linked ketal and
(ii) reacting a fragrance molecule having one or more alcohol moieties with the PEG linked ketal in a presence of tetrabutylammonium tribromide (TBAB) and trimethyl orthoformate 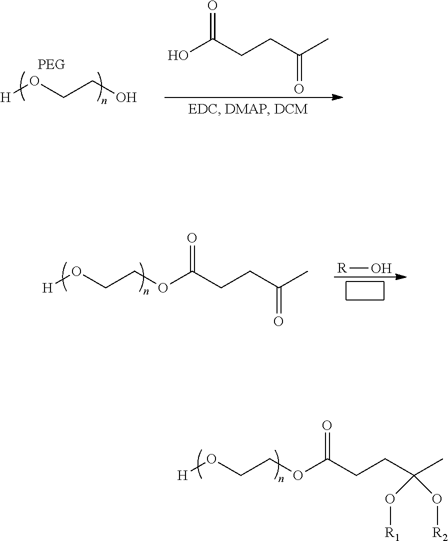
wherein R₁, R₂ are either one or more of mono- or poly-alcohol derivatives.

10. The process of claim 9 wherein at least one of R₁ and R₂ are diols or polyols.

11. The process of claim 9, wherein R₁ and R₂ are selected from the group consisting of one or more of: provitamins, vitamins, pain relief agents, and small molecule pharmaceuticals which are as distinguished from mono-alcohol or poly-alcohol containing fragrance moieties.

12. The process of claim 9, wherein each of the R₁, R₂ groups may be unique and different from any other or all of these groups within the PEG linked ketal molecule.

13. A process for decomplexation of fragranced PEG linked ketal compounds and consequent release of one or more fragrance molecules having at least one alcohol moiety, via an acid hydrolysis process step, as shown below: 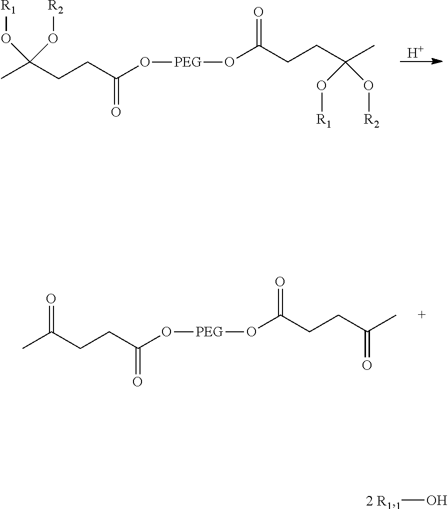
in which process, the PEG linkaged ketal is subjected to an environment where the conditions are acidic, or wherein the PEG linkaged ketal is contacted with an acid, whereby the PEG linkaged ketal structure undergoes an acid catalysis which releases the one or more fragrance molecules having at least one alcohol moiety and, in some cases, also acetone as a by-product.

14. A process for decomplexation of PEG-ketal compounds and consequent release of one or more fragrance molecules having at least one alcohol moiety, via an acid hydrolysis process step, as shown below: 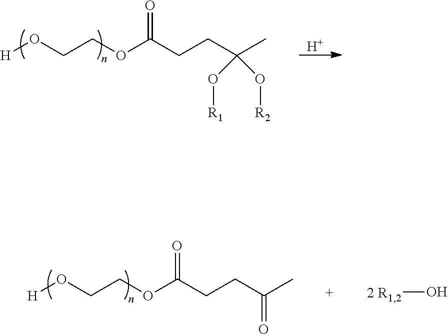
in which process, the PEG linkaged ketal is subjected to an environment where the conditions are acidic, or wherein the PEG linkaged ketal is contacted with an acid, whereby the PEG linkaged ketal structure undergoes an acid catalysis which releases the one or more fragrance molecules having at least one alcohol moiety and, in some cases, also acetone as a by-product.

15. The process of claim 13 or 14, wherein PEG-ketal compounds by coming into contact with the epidermis of a mammalian body, or with saliva or other bodily fluid which has a pH of less than 7.

16. The process of claim 15, wherein the PEG-ketal compounds form part of a topically applied composition which is applied to the epidermis of mammalian body.

17. The process of claim 16, wherein the PEG-ketal compounds form part of an orally ingestible composition.

18. The process of claim 15, wherein the PEG-ketal compounds form part of a treatment composition for inanimate surfaces, or an aerosolizable composition.

19. A polyketal according to the structure II: 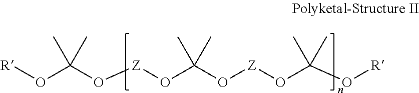
wherein
R′ is a terminated independent hydrogen, or R′ is a C₁-C₁₀ alkyl, or C₅-C₁₀ cycloalkyl and wherein R′—OH is a C₁-C₁₀ alkyl alcohol or a C₅-C₁₀ cycloalkyl alcohol and R′ of R′—OH is not H that is optionally substituted with an oxygen in the ring and/or further optionally substituted with one or more aryl groups or combinations thereof;
Z is a C₁-C₁₀ alkyl, and/or a C₅-C₆ cycloalkyl including cyclohexane, that is optionally substituted with an oxygen in the ring and/or further optionally substituted with one or more aryl groups such that O—Z—O is an ester group in that it is derived from an acid in which at least one —OH group is replaced by an —O-alkyl or —O-aryl group
and where n is in a range between 1-200.

20. A polyketal of claim 18, wherein R′—OH are selected from one or more of a group of substituted alcohols consisting of;
hydroxy cinnamyl alcohol;
rhodinol;
anisyl alcohol;
alpha-terpinol;
nerol;
maltol;
leaf alcohol;
ebanol;
dihydromercinol;
hydroxycitronellal;
lavender ketone;
raspberry ketone;
dimetol;
phenyl ethyl alcohol;
alpha-methylcinnamic alcohol;
linalool oxide;
acetoin;
isopentyl alcohol;
isoamyl alcohol;
2-phenyl methanol;
4-allyl-2-methoxyphenol (eugenol);
3-(2-bornyloxy)-2-methyl-1-propanol;
2-tert-butylcyclohexanol;
4-tert-butylcyclohexanol;
benzyl alcohol;
1-decanol;
9-decen-1-ol;
dihydroterpineol;
2,4-dimethyl-4-cyclohexen-1-yl methanol;
2,4-dimethylcyclohexyl methanol;
2,6-dimethyl-2-heptanol;
2,6-dimethyl-4-heptanol;
3a,4,5,6,7,7a-hexahydro-2,4-dimethyl-4,7-methano[1H] inden-5-ol;
3,7-dimethyl-1,6-nonadien-3-ol;
2,6-dimethyl-2,7-octadien-6-ol (linalool);
cis-3,7-dimethyl-2,6-octadien-1-ol (nerol);
trans-3,7-dimethyl-2,6-octadien-1-ol (geraniol;
3,7-dimethyl-1,7-octanediol;
3,7-dimethyl-1-octanol (tetrahydrogeraniol);
2,6-dimethyl-2-octanol (tetrahydromyrcenol);
3,7-dimethyl-3-octanol (tetrahydrolinalool);
2,6-dimethyl-7-octen-2-ol (dihydromyrcenol);
3,7-dimethyl-6-octen-1-ol (citronellol);
2,2-dimethyl-3-(3-methylphenyl)-1-propanol;
2,2-dimethyl-3-phenyl-1-propanol, 2-ethoxy-4-methoxymethylphenol;
2-ethyl-4-(2,2,3-trimethyl-3-cyclopenten-1-yl)-2-buten-1-ol;
cis-3-hexen-1-ol, 4-(4-hydroxy-3-methoxyphenyl)-2-butanone;
1-hydroxy-2-(1-methyl-1-hydroxyethyl)-5-methylcyclohexane;
3-(hydroxymethyl)-2-nonanone;
4-(4-hydroxy-4-methylpentyl)-3-cyclohexene-1-carboxaldehyde;
isoborneol;
3-isocamphylcyclohexanol;
2-isopropenyl-5-methylcyclohexanol (isopulegol);
1-isopropyl-4-methylcyclohex-3-enol (terpinenol);
4-isopropylcyclohexanol, 1-(4-isopropylcyclohexyl) ethanol;
4-isopropylcyclohexylmethanol;
2-isopropyl-5-methylcyclohexanol (menthol);
2-isopropyl-5-methylphenol (thymol), 5-isopropyl-2-methylphenol (carvacrol);
2-(4-methyl-3-cyclohexenyl)-2-propanol (terpineol);
2-(4-methylcyclohexyl)-2-propanol (dihydroterpineol);
4-methoxybenzyl alcohol, 2-methoxy-4-methylphenol;
3-methoxy-5-methylphenol;
1-methoxy-4-propenylbenzene (anethol);
2-methoxy-4-propenylphenol (isoeugenol);
4-methyl-3-decen-5-ol;
2-methyl-6-methylene-7-octen-2-ol (myrcenol);
3-methyl-4-phenyl-2-butanol;
2-(2-methylphenyl) ethanol;
2-methyl-4-phenyl-1-pentanol;
3-methyl-5-phenyl-1-pentanol;
2-methyl-1-phenyl-2-propanol;
(1-methyl-(1,2,2-trimethylbicyclo[3.1.0]hex-3-ylmethyl) cyclopropyl) methanol;
3-methyl-4-(2,2,6-trimethylcyclohexen-1-yl)-2-butanol;
2-methyl-4-(2,2,3-trimethyl-3-cyclopenten-1-yl)-2-buten-1-ol;
(3-methyl-1-(2,2,3-trimethyl-3-cyclopentenyl)-3-cyclohexen-1-yl) methanol;
3-methyl-5-(2,2,3-trimethyl-3-cyclopenten-1-yl)-4-penten-2-ol;
2-methyl-2-vinyl-5-(1-hydroxy-1-methylethyl) tetrahydrofuran;
trans,cis-2,6-nonadienol;
1-nonanol;
nopol;
1,2,3,4,4a,5,6,7-octahydro-2,5,5-trimethyl-2-naphthol;
1-octanol;
3,4,5,6,6-pentamethyl-2-heptanol;
2-phenylethanol;
2-phenylpropanol;
3-phenylpropanol (hydrocinnamic alcohol);
3-phenyl-2-propen-1-ol (cinnamic alcohol);
4-(5,5,6-trimethylbicyclo[2.2.1]hept-2-yl) cyclohexan-1-ol;
3,5,5-trimethylcyclohexanol;
2,4,6-trimethyl-4-cyclohexen-1-ylmethanol;
5-(2,2,3-trimethyl-3-cyclopentenyl)-3-methylpentan-2-ol;
3,7,11-trimethyl-2,6,10-dodecatrien-1-ol (farnesol);
3,7,11-trimethyl-1,6,10-dodecatrien-3-ol (nerolidol);
3,5,5-trimethyl-1-hexanol (isononanol);
1-undecanol;
10-undecen-1-ol; and
vetiverol.

21. A process of producing fragrance functional polyketals of Structure II 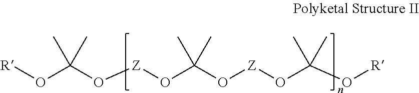
according to the reaction scheme illustrated: 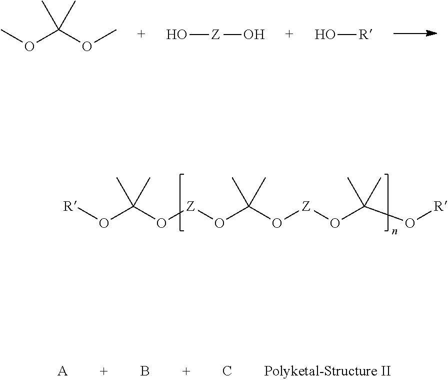
wherein a diol (B) is reacted with 2,2-dimethoxypropane (A), and a mono-alcohol fragrance (HO—R′) in p-toluene-sulfonic acid (C) to provide a polyketal of Structure II;
wherein R′ is a terminated independent hydrogen, or R′ is a C₁-C₁₀ alkyl, or C₅-C₁₀ cycloalkyl and wherein R′—OH is a C₁-C₁₀ alkyl alcohol or a C₅-C₁₀ cycloalkyl alcohol and R′ of R′—OH is not H that is optionally substituted with an oxygen in the ring and/or further optionally substituted with one or more phenyl groups;
Z is a C₁-C₁₀ alkyl, and/or a C₅-C₆ cycloalkyl including cyclohexane, that is optionally substituted with an oxygen in the ring and/or further optionally substituted with one or more aryl groups such that O—Z—O is an ester group in that it is derived from an acid in which at least one —OH group is replaced by an —O-alkyl or —O-aryl group
and where n is in a range between 1-200.

22. A process of acid catalysis of a polyketal with an acid at a pH of below 7 according to the following reaction scheme; 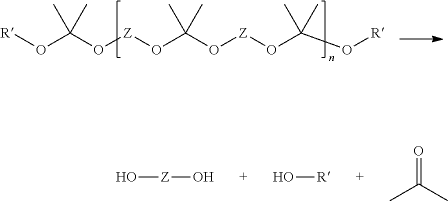
wherein R′ of said substituents are H, C₁-C₁₀ alkyl, and/or a C₅-C₁₀ cycloalkyl and wherein R′—OH is a C₁-C₁₀ alkyl alcohol or a C₅-C₁₀ cycloalkyl alcohol and R′ of R′—OH is not H that is optionally substituted with an oxygen in the ring and/or further optionally substituted with one or more aryl groups;
and wherein;
Z of said substituents is a C₁-C₁₀ alkyl, and/or a C₅-C₆ cycloalkyl including cyclohexane, that is optionally substituted with an oxygen in the ring and/or further optionally substituted with one or more aryl groups such that O—Z—O is an ester group in that it is derived from an acid in which at least one —OH group is replaced by an —O-alkyl or —O-aryl group.

23. The process of claim 21, wherein the polyketals reach a higher weight average molecular weight by reflux at 100 degrees Celsius to boil off methanol, addition of 2,2-dimethoxypropane and benzene every 2 hours for 12 hours, and use of a 5 A molecular sieve to capture excess methanol.

24. The process of claim 23, wherein said polyketals reach a weight average molecular weight of greater than 1000 g/mol and exhibit a polydispersity index (PDI) of less than 3.00.

25. The polyketal of claim 1, wherein the PEGs are substituted by polymers containing alcohol containing functionalities from one or more of a group consisting of: polysaccharides, starch, modified starch, cellulose, hydroxypropyl methylcellulose, hydroxyethyl cellulose, monosaccharides, lipids, polyester, polyamides, polyvinyl alcohol, polynucleotides, polyacetals, and polyurethanes.
